(19) United States
(12) Patent Application Publication
(10) Pub. No.: US 2022/0362225 A1
(43) Pub. Date: Nov. 17, 2022

(54) AEROSOL FORMULATION, CANISTER, AND INHALER CONTAINING THE FORMULATION, AND METHOD OF USE

(71) Applicant: KINDEVA DRUG DELIVERY L.P., Woodbury, MN (US)

(72) Inventors: Alexander D. Slowey, Nottingham (GB); Sarah E. Wrigglesworth, Leicestershire (GB)

(73) Assignee: KINDEVA DRUG DELIVERY L.P., Woodbury, MN (US)

(21) Appl. No.: 17/624,433

(22) PCT Filed: Mar. 18, 2020
(86) PCT No.: PCT/IB2020/052474

§ 371 Date: Jan. 3, 2022

Related U.S. Application Data
(60) Provisional application No. 62/873,388, filed on Jul. 12, 2019.

(51) Int. Cl. A61K 31/439; A61K 9/00; A61K 31/137; A61K 9/12; A61K 47/10; A61M 15/00

(52) U.S. Cl. CPC A61K 31/439; A61K 9/008; A61K 31/137; A61K 9/124; A61K 47/10; A61M 15/0066

(57) ABSTRACT

Pressurized canisters coated with poly(fluoroalkylene) polymer or a copolymer of poly(fluoralkylenes) and containing a formulation comprising one or more active pharmaceutical ingredients comprising umeclidinium or a salt thereof, vilanterol or a salt thereof, or a combination of two or more of the foregoing, poly(ethylene glycol), and a propellant, inhalers comprising the pressurized canister, as well as methods of using the pressurized canisters and inhalers.

### BACKGROUND

Aerosol formulations, particularly those containing one or more active pharmaceutical ingredients, are known in the art to also contain propellants and excipients.

### SUMMARY

A pressurized canister comprising can comprise a formulation. The formulation itself can comprise an active pharmaceutical ingredient comprising umeclidinium or a salt thereof; vilanterol or a salt thereof, or a combination of two or more of the foregoing; one or more propellants, and poly(ethylene glycol) having a number average molecular weight from about 50 to about 1,000. The interior of the pressurized canister can be coated with a poly(fluoroalkylene) polymer or a copolymer of poly(fluoralkylenes).

### DETAILED DESCRIPTION

Throughout this disclosure, singular forms such as “a,” “an,” and “the” are often used for convenience; however, the singular forms are meant to include the plural unless the singular alone is explicitly specified or is clearly indicated by the context. When the singular alone is called for, the term “one and only one” is typically used.

Some terms in this disclosure are defined below. Other terms will be familiar to the person of skill in the art, and should be afforded the meaning that a person of ordinary skill in the art would have ascribed to them.

The terms “common,” “typical,” and “usual,” as well as “commonly,” “typically,” and “usually” are used herein to refer to features that are often employed in the invention and, unless specifically used with reference to the prior art, are not intended to mean that the features are present in the prior art, much less that those features are common, usual, or typical in the prior art.

The term “active pharmaceutical ingredient” is intended to include both free compounds as well as pharmaceutically acceptable salts, hydrates, and solvates thereof. Salts of hydrates or solvates are also included, so long as they are pharmaceutically acceptable. When the name of a particular active pharmaceutical ingredient is used, it is likewise intended to include pharmaceutically acceptable salts, hydrates, and solvates thereof, as well as salts of hydrates or solvates so long as they are pharmaceutically acceptable. If the free compound or a particular salt, hydrate, solvate, or the like is specifically called for, it is identified as such in this disclosure.

The term “poly(ethylene glycol)” is used to refer to a polymer having the repeat unit —O—(CH₂)₂—. Depending on the way in which it is produced, poly(ethylene oxide). May have the same chemical structure, in which case it is encompassed by the term poly(ethylene glycol).

In this disclosure, the percent (%) concentration of components in a formulation is provided as a weight percent unless otherwise specified.

Fluoroalkanes are alkanes wherein at least some of the hydrogen atoms are replaced by fluoride.

Perfluoroalkanes are fluoroalkanes wherein essentially all of the hydrogen atoms are replaced by fluorine, but still allow the possibility that a small number of hydrogen atoms may be replaced by bromine or iodine instead of fluorine.

Fluoroalkanes and perfluoroalkanes may be referred to with carbon numbers to indicate the number of carbon atoms; in the event that a polymer or copolymer is referred to, the carbon numbers refer to the number of carbon atoms in the monomer or monomers from which the polymer was made.

“Trace amount” or “trace amounts” refers to an amount of a component that is unavoidably or unintentionally present, and the like, for example as amounts of contaminants, byproducts or a chemical reaction or industrial process (for example, filling and pressurizing a canister), minor components of industrially available materials, amounts that are not conveniently removable by common purification methods, and so forth. Trace amounts of materials in a formulation are limited to those amounts that do not have any appreciable effect on the properties of the formulation.

“Alcohol” refers to alcohol solvents or dispersants, typically ethanol but also including methanol, propanol, butanol, and the like. When the chemical moiety —OH is referred to, the term “alcohol moiety” is used.

Delivery of formulations containing active pharmaceutical ingredients by inhalation can be performed using inhalers with valves, such as metering valves. Metering valves are valves that regulate the amount of formulation that passes out of the inhaler and is delivered to the patient.

In order to deliver the desired dose of active pharmaceutical ingredient to the patient, it is not only necessary that the same amount of formulation pass through the valve each time the valve is opened to deliver a dose, but it is also necessary that the concentration of active pharmaceutical ingredient in the formulation that passes out of the container, through the valve, and to the patient be the same as the concentration of active pharmaceutical ingredient in the container.

A problem recognized in this disclosure is that the concentration of active pharmaceutical ingredient in the formulation that passes through the valve can be significantly lower than the concentration of active pharmaceutical ingredient in the formulation that is in the container. Put another way, the amount of active pharmaceutical ingredients that would be expected to be released after a single actuation of a valve, which can be referred to as the theoretical dose, is equal to the concentration of the active pharmaceutical ingredients in the formulation that is present in the canister multiplied by the volume of formulation that is released in a single dose (which often corresponds to one actuation of the valve, but may correspond to two or more actuations in some cases). A problem recognized in this disclosure is that the actual, measured amount of active pharmaceutical ingredients in a dose can be significantly lower than the theoretical dose. A related problem recognized in this disclosure is that when the actual dose is significantly lower than the theoretical dose, a significant amount of active pharmaceutical ingredients can be wasted. Because the active pharmaceutical ingredients are typically the most expensive components of an inhaler, this waste represents a significant cost increase.

It should be noted that an acceptable solution to this problem need not make the actual dose equal to the theoretical dose. Instead, an acceptable solution would increase the ratio of the actual dose to the theoretical dose, or reduce the amount of active pharmaceutical ingredients that are wasted.

Another problem is that there are currently no acceptable metered dose inhalers of certain inhalable active pharmaceutical ingredients, in particular umeclidinium and vilanterol, and more particularly umeclidinium bromide and vilanterol trifenatate. A dry powder inhaler of umeclidinium bromide and vilanterol trifenatate is available from GlaxoSmithKline under the trade designation Anoro Ellipta. However, dry powder inhalers are not acceptable for all patients because they rely solely on the power of the patient's inhalation to deliver the drug, and many patients who require inhaled medicines are not able to inhale deeply enough or with enough power to receive drugs from a dry powder inhaler Thus, another problem to be solved relates to a metered dose inhaler, and pressurized canister therefor, that can be used to deliver umeclidinium or vilanterol or both, and particularly umeclidinium bromide and vilanterol trifenatate.

Briefly, a solution lies in the combination of a formulation having one or more active pharmaceutical ingredients, one or more propellants, and poly(ethylene glycol) having a number average molecular weight of about 50 to about 1,000 within a pressurized canister, wherein the interior of the pressurized canister is coated with a poly(fluoroalkylene) polymer or a copolymer of poly(fluoralkylenes). It has been surprisingly shown that when the foregoing is used the actual dose is much closer to the theoretical dose than with other excipient and pressurized canister coating combinations; it is also an improvement over excipient-free formulations.

The one or more active pharmaceutical ingredients can in principle be any active pharmaceutical ingredients, but most typically selected from active pharmaceutical ingredients that are suitable for delivery by inhalation. In some cases, the one or more active pharmaceutical ingredients can include one or more of albuterol, levabuterol, formoterol, glycopyrrate, ciclesonide, mometasone, fluticasone, formaterol, ipratropium, beclomethasone, epinephrine, tiotropium, nicotine, umeclidimium, and vilanterol. In some cases, the one or more active pharmaceutical ingredients can include one or more active pharmaceutical ingredients that have at least one amine moiety, at least one alcohol moiety, or at least one amine moiety and at least one alcohol moiety. In some cases, the one or more active pharmaceutical ingredients can include one or more active pharmaceutical ingredients that have at least one amine moiety and at least one alcohol moiety. In specific cases, the one or more active pharmaceutical ingredients comprise umeclidinium or vilanterol. In more specific cases, the one or more active pharmaceutical ingredients comprise umeclidinium and vilanterol. The one or more active pharmaceutical ingredients can be present in the formulation either as a suspension, typically of micronized particles of the one or more active pharmaceutical ingredients, or they can be dissolved in the formulation. In most cases, the one or more active pharmaceutical ingredients are suspended in the formulation. The concentration of any of the one or more active pharmaceutical ingredients in the formulation can be any concentration that provides a suitable dosage of the particular active pharmaceutical ingredient to the patient.

The propellant can be any propellant suitable for use in an inhaler, such as a metered dose inhaler, but is most commonly a hydrofluorocarbon propellant. Examples include HFC-227, HFC-152a, and HFC-134a, as well as combinations of two or more of the foregoing. HFC-227 is most commonly used.

The poly(ethylene glycol) has a number average molecular weight of about 50 to about 1,000. Most commonly, the number average molecular weight is 50 or greater, 75 or greater, 100 or greater 125 or greater, 150 or greater, 175 or greater, 200 or greater, 225 or greater, 250 or greater, or 275 or greater. Most commonly, the number average degree of polymerization is 1,000 or less, 900 or less, 800 or less, 750 or less, 700 or less, 650 or less, 600 or less, 550 or less, 500 or less, 475 or less, 450 or less, 425 or less, 400 or less, 375 or less, 350 or less, or even 325 or less. In one example, the number average molecular weight is about 300. In a particular case PEG-300 is used.

The concentration of the poly(ethylene glycol) in the formulation can be any amount sufficient to provide an actual dose that is nearer to the theoretical dose than would be obtained without the poly(ethylene glycol). Most commonly, the poly(ethylene glycol) will be present in a concentration of about 0.001% to about 1% of the formulation. Exemplary concentrations can be about 0.001% or greater, 0.005% or greater, 0.0075% or greater, 0.01% or greater, 0.02% or greater, 0.03% or greater, 0.04% or greater, 0.05% or greater, 0.06% or greater, 0.07% or greater, 0.08% or greater, or 0.09% or greater. Exemplary concentrations can be 1% or less, 0.9% or less, 0.8% or less, 0.7% or less, 0.6% or less, 0.5% or less, 0.45% or less, 0.4% or less, 0.35% or less, 0.3% or less, 0.29% or less, 0.28% or less, 0.27% or less, 0.26% or less, 0.25% or less, 0.24% or less, 0.23% or less, 0.22% or less, 0.21% or less, 0.2% or less, 0.19% or less, 0.18% or less, 0.17% or less, 0.16% or less, 0.15% or less, 0.14% or less, 0.13% or less, 0.12% or less, or 0.11% or less. A concentration can be from about 0.005% to about 0.5%. Ina particular case the concentration can be 0.01%.

Particular formulation can be free of more than trace amounts of components other than the one or more active pharmaceutical ingredients, the one or more propellants, and the poly(ethylene glycol). Some particular formulations are free of more than trace amounts of alcohols, particularly ethanol. Some particular formulations are free of more than trace amounts of water. Some particular formulations are free of more than trace amounts of water and alcohol, such as ethanol. Some particular formulations are free of more than trace amounts of surfactant (other than the poly(ethylene glycol)).

The canister coating is a poly(fluoroalkylene) polymer or a copolymer of poly(fluoralkylenes) is a copolymer of poly(fluoroalkylenes). The fluroalkylenes used in the polymer or copolymer are typically C2-C10 fluoroalkanes. The fluoroalkylenes used I the polymer or copolymer are most commonly Most commonly, a copolymer of poly(fluoroalkylenes) is used. A typical copolymer is a copolymer of a C2-C4 fluoroalkylene and a C3-C6 fluoroalylene. Most commonly, the copolymer is a copolymer of hexafluropropylene and tetrafluoroethylene. Such copolymers are sometimes referred to in the art as FEP.

An inhaler can contain a pressurized canister as described herein. Typically, inhalers will also include a valve. The valve is typically in communication with an actuator such that when the actuator is actuated at least a part of the formulation is released from the inhaler Most often the valve is a metering valve. Metering valves, sometimes referred to as metered dose valves, are known, and any suitable metering valve can be used. Suitable metering valves include those that are able to release a volume of formulation with a pharmaceutically effective amount of active pharmaceutical agent, and that do not chemically interact with the components of the formulation. At least a portion of the metering valve can be coated with a poly(fluoroalkylene) polymer or a copolymer of poly(fluoralkylenes), such as any of the poly(fluoroalkylene) polymer or a copolymer of poly(fluoralkylenes) described above with respect to the coating of the canister. This is not required, and other coatings or in some cases no coatings can also be used.

A method of actuating an inhaler, such as any inhaler described herein, is disclosed. The method can comprise actuating an actuator for a sufficient period of time to release at least a portion of the formulation from the pressurized canister. In some embodiments in which umeclidinium bromide is employed, a single actuation of the inhaler releases 55.2-74.8 micrograms of umeclidinium bromide from the inhaler. In some embodiments in which umeclidinium is employed, a single actuation of the inhaler releases 58.5-71.5 micrograms of umeclidinium bromide from the inhaler. In some in which vilanterol trifenatate is employed, a single actuation of the inhaler releases 29.8-40.3 micrograms of vilanterol trifenatate from the inhaler. In some embodiments in which vilanterol trifenatate is employed, a single actuation of the inhaler releases 31.5-39.0 micrograms of vilanterol trifenatate from the inhaler. It should be noted that the term “a single actuation” should not be understood to require not every actuation releases the above-mentioned amounts of pharmaceutically active agent or agents. For example, one or more initial or priming actuation or actuations may release less than the specified amounts. It should also be understood that the foregoing embodiments are not the only possible embodiments, and that other amounts of active agents can be released if desired.

A method of administering a formulation, such as the formulations disclosed herein, is disclosed. The method can comprise actuating the actuator for a sufficient time to release at least a portion of a formulation as described herein from the pressurized canister and inhaling at least a portion of the formulation. The method can include administering a pharmaceutically acceptable amount of umeclidinium, vilanterol, or a combination thereof. In some embodiments in which umeclidinium bromide is employed, a single actuation of the inhaler releases 55.2-74.8 micrograms of umeclidinium bromide from the inhaler. In some embodiments in which umeclidinium is employed, a single actuation of the inhaler releases 58.5-71.5 micrograms of umeclidinium bromide from the inhaler. In some in which vilanterol trifenatate is employed, a single actuation of the inhaler releases 29.8-40.3 micrograms of vilanterol trifenatate from the inhaler. In some embodiments in which vilanterol trifenatate is employed, a single actuation of the inhaler releases 31.5-39.0 micrograms of vilanterol trifenatate from the inhaler. It should be noted that the term “a single actuation” should not be understood to require not every actuation releases the above-mentioned amounts of pharmaceutically active agent or agents. For example, one or more initial or priming actuation or actuations may release less than the specified amounts. It should also be understood that the foregoing embodiments are not the only possible embodiments, and that other amounts of active agents can be delivered if desired.

A method of actuating an inhaler, such as any inhaler described herein, is disclosed. The method can comprise actuating an actuator for a sufficient period of time to release at least a portion of the formulation from the pressurized canister. In some embodiments in which umeclidinium bromide is employed, a single actuation of the inhaler releases a metered dose of 63.0-85.3 micrograms of umeclidinium bromide from the valve. In some embodiments in which umeclidinium bromide is employed, a single actuation of the inhaler releases 66.8-81.6 micrograms of umeclidinium bromide from the valve. In some embodiments in which vilanterol trifenatate is employed, a single actuation of the inhaler releases a metered dose of 34.0-46.0 micrograms of vilanterol trifenatate from the valve. In some embodiments in which vilanterol trifenatate is employed, a single actuation of the inhaler releases 36.0-44.0 micrograms of vilanterol trifenatate from the valve. It should be noted that the term “a single actuation” should not be understood to require not every actuation releases the above-mentioned amounts of pharmaceutically active agent or agents. For example, one or more initial or priming actuation or actuations may release less than the specified amounts. It should also be noted that other amounts of actives can be released from the valve is desired

A method of administering a formulation, such as the formulations disclosed herein, is disclosed. The method can comprise actuating the actuator for a sufficient time to release at least a portion of a formulation as described herein from the pressurized canister and inhaling at least a portion of the formulation. The method can include administering a pharmaceutically acceptable amount of umeclidinium, vilanterol, or a combination thereof. When the method is performed with umeclidinium bromide, a single actuation of the inhaler releases a metered dose of 63.0-85.3 micrograms of umeclidinium bromide from the valve. In some embodiments of the method, a single actuation of the inhaler releases 66.8-81.6 micrograms of umeclidinium bromide from the valve. When the method is employed with vilanterol trifenatate, a single actuation of the inhaler releases a metered dose of 34.0-46.0 micrograms of vilanterol trifenatate from the valve. In some embodiments of the method, a single actuation of the inhaler releases 36.0-44.0 micrograms of vilanterol trifenatate from the valve. It should be noted that the term “a single actuation” should not be understood to require not every actuation releases the above-mentioned amounts of pharmaceutically active agent or agents. For example, one or more initial or priming actuation or actuations may release less than the specified amounts. It should also be noted that other amounts of active can be released if desired.

For any of the methods of actuating an inhaler and methods of administering a formulation that are described above, a single actuation of the inhaler can release 0.11-0.15 micromoles of umeclidinium or a pharmaceutically acceptable salt thereof from the inhaler. In some embodiments of the methods, a single actuation of the inhaler releases 0.12-0.14 micromoles of umeclidinium or a pharmaceutically acceptable salt thereof from the inhaler.

For any of the methods of actuating an inhaler and methods of administering a formulation that are described above, a single actuation of the inhaler can release 0.038-0.052 micromoles of vilanterol or a pharmaceutically acceptable salt thereof from the inhaler. In some embodiments of the methods, a single actuation of the inhaler releases 0.040-0.050 micromoles of vilanterol or a pharmaceutically acceptable salt thereof from the inhaler.

For any of the methods of actuating an inhaler and methods of administering a formulation that are described above, a single actuation of the inhaler can release a metered dose of 0.12-0.17 micromoles of umeclidinium or a pharmaceutically acceptable salt thereof from the valve. In some embodiments of the methods, a single actuation of the inhaler releases 0.13-0.16 micromoles of umeclidinium or a pharmaceutically acceptable salt thereof from the valve.

For any of the methods of actuating an inhaler and methods of administering a formulation that are described above, a single actuation of the inhaler can release a metered dose of 0.041-0.060 micromoles of vilanterol or a pharmaceutically acceptable salt thereof from the valve. In some embodiments of the methods, a single actuation of the inhaler releases 0.046-0.056 micromoles of vilanterol or a pharmaceutically acceptable salt thereof from the valve.

For any of the methods of actuating an inhaler and methods of administering a formulation that are described above, a single actuation of the inhaler can release 46.7-63.3 micrograms of umeclidinium from the inhaler. In some embodiments of the methods, a single actuation of the inhaler releases 49.5-60.5 micrograms of umeclidinium from the inhaler.

For any of the methods of actuating an inhaler and methods of administering a formulation that are described above, a single actuation of the inhaler can release 18.7-25.3 micrograms of vilanterol from the inhaler. In some embodiments of the methods, a single actuation of the inhaler releases 19.8-24.2 micrograms of vilanterol from the inhaler.

For any of the methods of actuating an inhaler and methods of administering a formulation that are described above, a single actuation of the inhaler can release a metered dose of 53.0-71.9 micrograms of umeclidinium from the valve. In some embodiments of the methods, a single actuation of the inhaler releases 56.2-68.8 micrograms of umeclidinium from the valve.

For any of the methods of actuating an inhaler and methods of administering a formulation that are described above, a single actuation of the inhaler can release a metered dose of 21.2-28.8 micrograms of vilanterol from the valve. In some embodiments of the methods, a single actuation of the inhaler releases 22.5-27.5 micrograms of from the valve.

### EXAMPLES

1,1,1,2-tetrafluoroethane (HFC-227) was obtained from Mexichem UK (Runcorn, UK). Vilanterol trifenatate and umeclidinium bromide were obtained from Hovione (Loures, Portugal). Poly(ethylene glycol) having an average molecular weight of 300 (PEG 300) was obtained from the Sigma-Aldrich Company (St. Louis, Mo.).

### Example 1

Each metered dose inhaler (MDI) was prepared using a 16 mL aluminum canister coated with FEP (IntraPac International, Mooresville, N.C., USA); a 63 microliter 3M retention type valve with a PBT (polybutylene terephthalate) stem and EPDM (ethylene-propylene diene terpolymer elastomer) diaphragm seal (3M Corporation); and an actuator with a 0.25 mm exit orifice diameter and 0.8 mm jet length. The bottle emptier, tank, spring, and ferrule components of the valves were coated with a fluoropolymer coating according to the general process described in Example 2 of U.S. Patent Application Publication 2017/0152396 A1, Jinks et al. The formulation used was umeclidinium bromide (1.237 mg/mL), vilanterol trifenatate (0.667 mg/mL), and PEG 300 (0.01 weight percent) in HFC-227 propellant. In the process, the HFC-227 propellant was dispensed into a batching vessel maintained at negative 60° C. and then PEG 300 was added to the vessel and dispersed to form a mixture. Umeclidinium bromide and vilanterol trifenatate were then added to the vessel and the mixture was high shear mixed to create a homogenous suspension (Silverson mixer, Silverson Machine Ltd., Chesham, UK). The canisters were cold filled with the suspension using a timer controlled solenoid valve. The inhalers were stored for 6 weeks under ambient conditions prior to testing.

Each finished inhaler was prepared to deliver targeted amounts of 74.2 micrograms umeclidinium bromide ex-valve per actuation and 40 micrograms of vilanterol trifenatate ex-valve per actuation.

The targeted delivery amounts were also calculated to represent the corresponding amount (micrograms) of umeclidinium delivered and the corresponding amount of vilanterol delivered. The calculated amounts were determined by the following equations (MW is an abbreviation for molecular weight):

umeclidinium (□g/actuation)=[umeclidinium bromide(□g/actuation)]×[MW umeclidinium÷MW umeclidinium bromide]

vilanterol (□g/actuation)=[vilanterol trifenatate (□g/actuation)]×[MW vilanterol÷MW vilanterol trifenatate]

Based on the equations, each finished inhaler was calculated to deliver targeted amounts of 62.5 micrograms umeclidinium ex-valve per actuation and 25 micrograms of vilanterol ex-valve per actuation.

### Comparative Example A

MDIs were prepared using the same method as described in Example 1 with the exception that the PEG 300 was not included in the formulation.

### Example 3. Next Generation Impactor (NGI) Assay

Each MDI was evaluated using a Next Generation Impactor Instrument (MSP Corporation, Shoreview, Minn.). For each test, an MDI was attached to the throat component (Emmace anatomical throat, Emmace Consulting, Lund, Sweden) of the NGI instrument and actuated six times into the instrument. Prior to each actuation the MDI was vigorously shaken. Immediately prior to attachment, the MDI was primed by actuating eight times. Prior to each priming shot the MDI was vigorously shaken. The flow rate through the instrument during testing was regulated at 30 L/minute. The test sample (umeclidinium bromide and vilanterol trifenatate) deposited on the valve stem, actuator, throat assembly (Emmace anatomical throat), individual uncoated collection cups 1-7, micro-orifice collector (MOC), and final filter component was collected by rinsing each individual component with a known volume of collection solvent (30 mL of collection solvent used for the throat assembly and 10 mL of collection solvent used for all of the other individual components). The collection solvent was sodium dodecyl sulfate (SDS, 10 mM) in 60:40 (volume/volume) acetonitrile:50 mM NH₄OAc, pH 5.5. The recovered samples were then analyzed for sample content using an HPLC assay with references to known standards. An Agilent 1100 HPLC instrument (Agilent Technologies, Santa Clara, Calif.) with a UV detector (220 nm) and a symmetry shield RP-18, 4.6 mm by 150 mm (3.5 micron) column (25° C. column temp) was used. Fresh collection solvent was used as the mobile phase. The injection volume was 50 microliters and the flow rate was 1.0 mL/minute.

The sample content measured for each component was summed to give a total value for recovered sample and then the total amount of test sample (umeclidinium bromide and vilanterol trifenatate) recovered per actuation was calculated. Three individual MDIs (n=3) were tested and the results are presented as the mean values (Tables 1 and 2). In Tables 1 and 2, the amounts of umeclidinium and vilanterol recovered (micrograms/actuation) are reported using the equations described in Example 1.

TABLE 1

| | Umeclidinium | | |
|---|---|---|---|
| | Recovered | Calculated | Percentage |
| | (micrograms/ | Umeclidinium | of |
| | actuation) | Delivered | Recovered |
| | with standard | (micrograms/ | versus |
| | deviation (SD) | actuation) | Calculated |
| Example 1 | 58.4 | 62.5 | 93.4 |
| | (SD = 2.3) | | |
| Comparative | 50.5 | 62.5 | 80.1 |
| Example 1 | (SD = 7.8) | | |

TABLE 2

| | Vilanterol | | |
|---|---|---|---|
| | Recovered | Calculated | |
| | (micrograms/ | Vilanterol | Percentage |
| | actuation) | Delivered | Recovered |
| | with standard | (micrograms/ | versus |
| | deviation (SD) | actuation) | Calculated |
| Example 1 | 23.1 | 25.0 | 92.4 |
| | (SD = 0.9) | | |
| Comparative | 20.1 | 25.0 | 80.4 |
| Example 1 | (SD = 3.2) | | |

### Example 4. Assay for Measuring Deposition of Umeclidinium and Vilanterol on Canisters

MDIs were prepared and stored as in Example 1 and Comparative Example 1. For the deposition assay, each MDI was actuated 45 times and then the canister with valve assembly was removed from the actuator housing. The canister was chilled by immersion in a liquid nitrogen bath for about one minute and the valve assembly was removed from the canister. The formulation was then poured from the canister into a separate receiving flask. The open canister was maintained at ambient conditions for 30 minutes to allow the canister to warm to ambient temperature (20-21° C.). The canister was then placed in a borosilicate glass boiling tube and 50 mL of the collection solvent (described in Example 3) was added to the tube. The tube was capped and then sonicated for 2 minutes followed by vigorous shaking by hand for one minute. An aliquot of the collection solvent was analyzed for sample content (using an HPLC assay with references to known standards). An Agilent 1100 HPLC instrument (Agilent Technologies, Santa Clara, Calif.) with a UV detector (220 nm) and a symmetry shield RP-18, 4.6 mm-150 mm column (25° C. column temp) was used. Fresh collection solvent was used as the mobile phase. The flow rate was 1.0 mL/minute. The amounts of umeclidinium and vilanterol recovered from the canisters are reported in Table 3. Three individual MDIs (n=3) were tested and the results are presented as the mean values.

The results in Table 3 are reported based on the following equations (MW is an abbreviation for molecular weight):

umeclidinium recovered (□g)=[umeclidinium bromide recovered (□g)]×[MW umeclidinium÷MW umeclidinium bromide]

vilanterol recovered (□g)=[vilanterol trifenatate recovered (□g)]×[MW vilanterol÷MW vilanterol trifenatate]

TABLE 3
Canister Deposition Assay

| | Umeclidinium | Vilanterol |
|---|---|---|
| | Recovered | Recovered |
| | from Canister | from Canister |
| | [micrograms | [micrograms |
| | with standard | with standard |
| | deviation (SD)] | deviation (SD)] |
| Example 1 | 606.7 | 238.3 |
| | (SD = 109.2) | (SD = 44.7) |
| Comparative | 1105.9 | 440.0 |
| Example 1 | (SD = 7.8) | (SD = 85.9) |

1. A pressurized canister comprising a formulation, the formulation comprising
an active pharmaceutical ingredient comprising umeclidinium or a salt thereof, vilanterol or a salt thereof, or a combination of two or more of the foregoing;
one or more propellants, and
poly(ethylene glycol) having a number average molecular weight from about 50 to about 1,000; wherein
the interior of the pressurized canister is coated with a poly(fluoroalkylene) polymer or a copolymer of poly(fluoralkylenes).

2. The pressurized canister of claim 1, wherein the one or more active pharmaceutical ingredients comprises umeclidinium bromide or vilanterol trifenatate.

3. The pressurized canister of claim 1, wherein the poly(ethylene glycol) has a number average molecular weight from about 50 to about 500.

4. The pressurized canister of claim 1, wherein the poly(ethylene glycol) has a number average molecular weight from about 100 to about 500.

5. The pressurized canister of claim 1, wherein the poly(ethylene glycol) is about 0.001% to about 1% by weight of the formulation.

6. The pressurized canister of claim 1, wherein the poly(ethylene glycol) is about 0.05% to about 0.5% by weight of the formulation.

7. The pressurized canister of claim 1, wherein the poly(fluoroalkylene) polymer or a copolymer of poly(fluoralkylenes) is a copolymer of poly(fluoroalkylenes).

8. The pressurized canister of claim 7, wherein the copolymer of poly(fluoroalkylenes) is a copolymer of a C2-C4 fluoroalkylene and a C3-C6 fluroalkylene.

9. The pressurized canister of claim 8, wherein the copolymer of poly(fluroalkylenes) is a copolymer of hexafluoropropylene and tetrafluoroethylene.

10. The pressurized container of claim 1, wherein the formulation contains no more than a trace amount of any component other than the one or more active pharmaceutical ingredients, the one or more propellants, and the poly(ethylene glycol).

11. The pressurized container of claim 10, further comprising a trace amount of water, alcohol, or water and alcohol.

12. The pressurized canister of claim 1, wherein the umeclidinium or a salt thereof, vilanterol or a salt thereof are present in the form of a suspension.

13. An inhaler comprising the pressurized canister of claim 1.

14. The inhaler of claim 13, wherein the inhaler further comprises a valve and an actuator in communication with the valve.

15. The inhaler of claim 14, wherein the valve is a metering valve.

16. The inhaler of claim 13, wherein at least a portion of the metering valve is coated with the poly(fluoroalkylene) polymer or a copolymer of poly(fluoralkylenes).

17. A method of actuating an inhaler comprising actuating an actuator of the inhaler of claim 13 for a sufficient time to release at least of a portion of the formulation from the pressurized canister.

18. A method of administering a formulation comprising actuating an actuator of the inhaler of claim 13 fora sufficient time to release at least a portion of the formulation from the pressurized container; and inhaling at least a portion of the formulation.

19-20. (canceled)

21. The method of claim 18, wherein a single actuation of the inhaler releases 55.2-74.8 micrograms of umeclidinium bromide from the inhaler.

22. The method of claim 18, wherein a single actuation of the inhaler releases 29.8-40.3 micrograms of vilanterol trifenatate from the inhaler.

23-30. (canceled)
